(19) United States
(12) Patent Application Publication
(10) Pub. No.: US 2019/0255312 A1
(43) Pub. Date: Aug. 22, 2019

(54) SYSTEM AND METHOD FOR APPLYING SUCTION DURING SURGERY

(71) Applicant: Jose Gustavo Espino, Alexandria, VA (US)

(72) Inventor: Jose Gustavo Espino, Alexandria, VA (US)

(21) Appl. No.: 16/278,975

(22) Filed: Feb. 19, 2019

Related U.S. Application Data
(63) Continuation-in-part of application No. 15/131,981, filed on Apr. 18, 2016, now abandoned.

(51) Int. Cl. A61M 39/10; A61M 1/00; A61B 1/00

(52) U.S. Cl. CPC A61M 39/105; A61B 1/317; A61B 1/00128; A61M 1/0086

(57) ABSTRACT

A method and system for applying suction during a surgical procedure that requires simultaneous application of suction to at least two surgical instruments or devices utilizes Y-configured suction hose arrangements that each includes a single body tube connected to a pair of extremities tubes by a Y-connector, the body tube being connected to a suction source and fluid collector, and the extremities tubes each being connected to one of the at least two surgical instruments or devices. The system and method of the invention is especially suitable for arthroscopic procedures, in which case the instruments or devices may include an arthroscopic shaver and/or cautery, a camera, and liquid waste collectors.

This application is a CIP of U.S. patent application Ser. No. 15/131,981, filed Apr. 18, 2016, incorporated herein by reference.

### BACKGROUND OF THE INVENTION

### 1. Field of the Invention

The invention relates to a system and method for connecting, and simultaneously applying suction to, multiple suction-requiring surgical instruments or devices during a surgical procedure.

The suction applying system and method of the invention may be utilized, for example, in an arthroscopic procedure.

### 2. Description of Related Art

A known suction system for surgeries in operation rooms consists of a final collector of fluids (canister, machines like the Nautilus™, etc.) that is connected to surgical instruments and other devices utilized during the surgery by regular sterile suction tubing made of PVC. The PVC tubing typically has a length of three meters. The instruments or other devices may include cannulas, scopes, and collector bags. Some types of surgeries require three or more regular suction tubes in order to connect each instrument. The requirement for so many tubes creates stress on the surgical team because they need to plug in and take care not only of the three tubes, but also connecting wires and cables for the instruments, to prevent the tubes, wires, and cables from getting tangled, bent, twisted and/or from being disconnected or falling, which would lead to contamination.

The principal function of the suction system has traditionally been to provide clear vision to the surgeon during the surgery by removing blood, bodily fluids, and other debris or by-products of the surgery, as well as liquids used when the wounds are being washed. Current surgical methods, however, may require simultaneous use of a relatively large number of instruments, such as cameras, shavers, and electrocautery instruments, each of which may require attachment to a suction tube.

Examples of surgical procedures that may rely multiple suction-requiring instruments and/or devices includes cystoscopic procedures, and minimally-invasive arthroscopic or laparoscopic surgeries. These types of procedures typically require at least two suction tube and two electrical cable connections, and sometimes as many as four regular suction tubes, resulting in the above-noted difficulties in managing the suction and cable connections.

### SUMMARY OF THE INVENTION

It is accordingly an objective of the invention to provide a surgical system and method that addresses problems caused by the need for simultaneous use of multiple suction tubes.

The objective is address by providing a suction system in which suction tubes for multiple suction-utilizing surgical instruments or devices are combined using “Y” connectors to connect pairs of instruments or devices with a suction source via a shared suction tube, thereby reducing the number of connections to the suction source by a factor of two.

The objective is also addressed by providing a method of performing surgery in which multiple instruments are connected to a suction source by “Y” connected suction tubes. The method of the invention may be applied to, by way of example and not limitation, arthroscopic and laparoscopic surgeries.

Advantages of the system and method of providing suction tubing in a “Y” configuration that replaces two regular suction tubes include the following:

1—saving time: fewer tubes being connected to the suction system, as well as less boxes to open, and less tangling between cables and tubes;

2—saving money: connection of relatively shorter tubes in a “Y” configuration to replace two longer tubes saves money; and

3—since suction tubes are disposable, the reduced overall length of suction tubing required for a surgical procedure reduces the amount of waste generated by the procedure.

The above-listed advantages are significant. For example, taking as a reference Inova® Alexandria hospital, which was reported to have 1000 arthroscopies in 2015, since each arthroscopic procedure required at least two suction tubes, each having a length of three meters +/−5 centimeters, the total number length of suction tubes required for the 1000 procedures amounted to more than 6000 meters (2000 suction tubes at three meters each), or about four miles of tubing.

If we were to use the suction tubing in “Y” two ways in the same hospital, with the same time and number of surgeries, the length of tubing could be reduced by at least one third, requiring only 4000 meters of PVC tubing. The difference of 2000 meters a year in just one hospital can make a big difference in cost and for the environment, and this is just for one hospital. The savings nationwide and worldwide are staggering.

### BRIEF DESCRIPTION OF THE DRAWINGS

FIG. 1 is a plan view of suction tubing having a “Y” configuration for use in the system and method of the invention.

FIG. 2 is a schematic illustration of an arthroscopic surgery system arranged in accordance with the principles of a preferred embodiment of the invention.

FIGS. 3 and 4 shows further aspects of the arthroscopic system of FIG. 2.

### DETAILED DESCRIPTION OF THE PREFERRED EMBODIMENTS

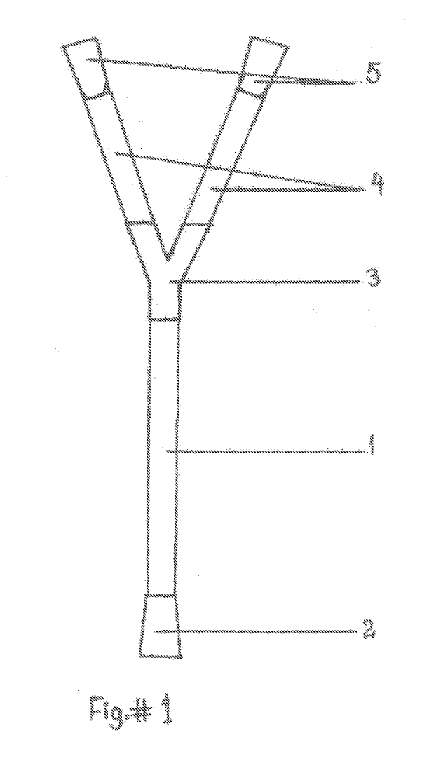

As shown in FIG. 1, the system of the invention utilizes suction tubing connected in a “Y” configuration. The suction hose or tubing arrangement includes two female connectors 5 arranged to be connected to suction-utilizing surgical instruments or devices, two PVC extremities tubes 4, a “Y”-shaped connector 3 made of polypropylene or a material stronger that PVC to avoid bending or twisting; a PVC body tube 1 in the surface; and a female connector 2 for connecting the body tube 1 to the suction system. The PVC body tube 1 and extremities tubes 4 may include optional linear ridges in the surface.

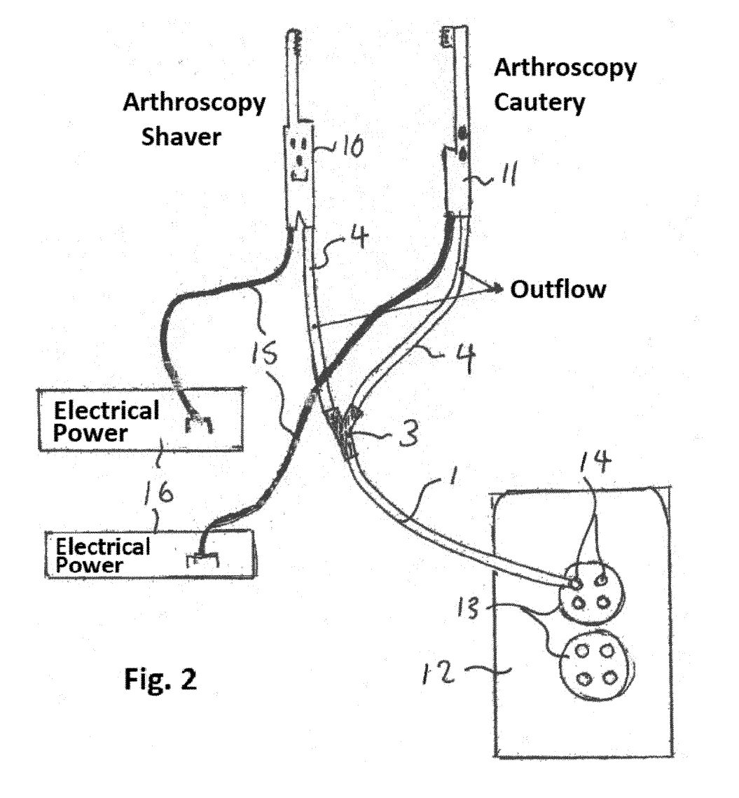

The “Y”-shaped hose or tubing arrangement of FIG. 1, which may come pre-packaged with the body and extremities tubes already connected to the Y-connector, is utilized in a surgical suction system such as the one shown in FIGS. 2-4, which is especially suitable for use in arthroscopic or laparoscopy surgeries. FIGS. 2-4 show an arthroscopic or laparoscopic surgery system, but the system may easily be adapted by those skilled in the art for use in other types of surgery that require multiple suction connections.

As illustrated in FIG. 2, the extremities tubes 4 are respectively coupled to an arthroscopy shaver 10 and arthroscopy cautery 11, each of which requires connection to a suction source. The suction source may take the form of the illustrated surgical waste management system 12, which has a number of canisters 13 and suction ports 14, one of which is connected to the female connector 2 of the Y tubing shown in FIG. 1. The shaver 10 and cautery 11 are each connected by a power cord 15 to an outlet 16, but because the suction tubes 4 are joined at the Y-connector 3, they are less likely to become entangled with the power cords 15 than would be the case with conventional separate suction tubes.

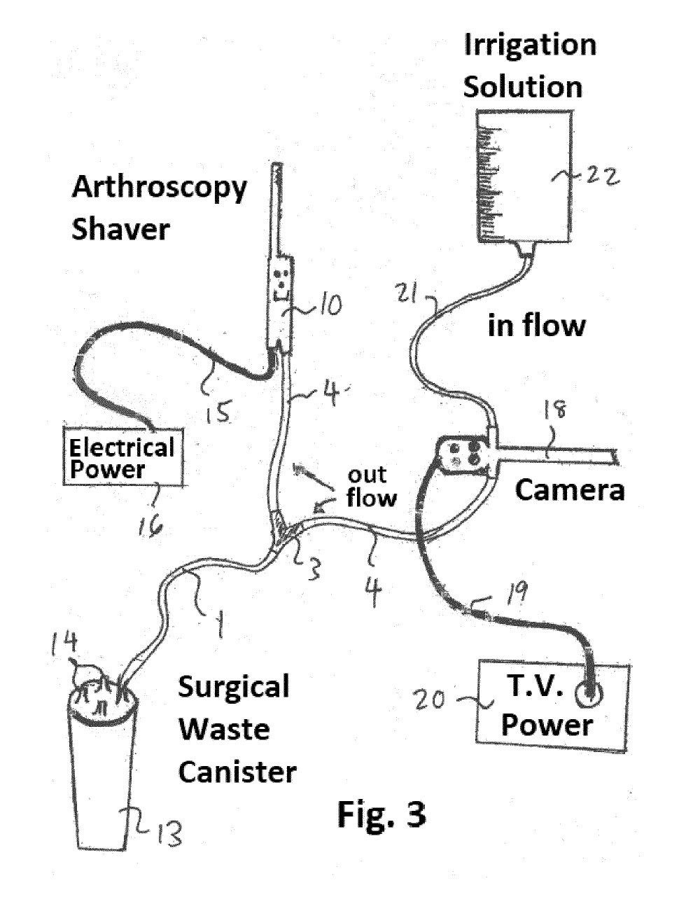

As illustrated in FIG. 3, one or both of the instruments shown in FIG. 2 may be replaced by alternative arthroscopic surgical instruments. In the set-up of FIG. 3, the cautery 11 is replaced by a camera 18, which is connected by a cable 19 to a monitor 20. The camera 18, which is coupled to a scope, may also be connected by tubing 21 to an irrigation solution source 22.

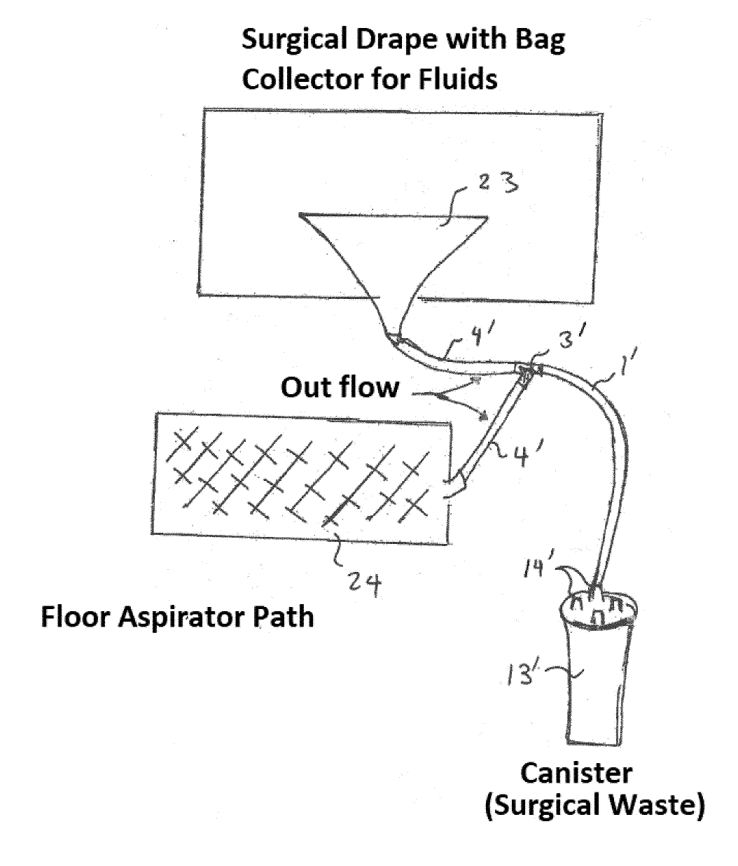

As illustrated in FIG. 4, a second body tube 1′, a second Y-connector 3′, a second pair of extremities tubes 4′, and a second canister 13′ with suction nozzles 14′ are used during the same surgical procedure as the corresponding equipment illustrated in FIGS. 2 and 3. Extremities tubes 4′ are connected to a surgical drape with a bag collector 23 for waste fluids flowing directly from the patient and a floor collector 24 for fluids not collected by the bag collector 23.

It will be appreciated that the arrangements of FIGS. 2-4 may be used with a variety of different surgical instruments, devices, implements, and fluid collectors that require connection to a suction source, for any type of procedure that requires simultaneous connection of multiple such instruments, devices, implements, or collectors including by way of example major arthroscopic procedures involving a knee, hip, or shoulder, minor arthroscopic procedures involving wrist, ankle, or hand, ACL and PCL reconstruction, cystoscopy, spinal surgery, and laparoscopic procedures. In addition, the arrangements of FIGS. 2-4 may require use of devices, such as light sources, that do not require connection to the suction source, but that may require cables that could become entangled with the suction tubing, and which would therefore benefit from the reduction in suction tubing provided by the present invention.

The embodiments illustrated in FIGS. 2-4 may be implemented by a method of providing suction during a surgical procedure, such as an arthroscopic procedure, that includes the steps of providing Y-configured suction hose having a body tube 1 connected to a pair of extremities tubes 4 by a Y-connector 3; connecting the body tube to a suction port 14 of a suction canister 13; connecting a first of the extremities tubes to a first surgical instrument or device; connecting the second of the extremities tubes to a second surgical instrument or device; and performing the surgical procedure while providing simultaneous suction to the first and second instruments or devices through the extremities tubes and the body tube. The method of the invention may further includes the steps of providing a second Y-configured suction hose having a body tube 1′ connected to a pair of extremities tubes 4′ by a Y-connector 3′; connecting the body tube 1′ to a suction port 14 of a suction canister 13; connecting a first of the extremities tubes 4′ to a third surgical instrument or device; connecting the second of the extremities tubes 4′ to a fourth surgical instrument or device; and performing the surgical procedure while providing simultaneous suction to the first, second, third, and fourth instruments or devices through the two pairs of extremities tubes and the two body tubes. The first and second surgical instruments or devices may include an arthroscopic shaver, and arthroscopic cautery, and/or a camera. The third and fourth surgical instruments or devices may include a bag collector and a floor collector for waste fluids.

Those skilled in the art will appreciate that the preferred embodiments described above are not intended to be limiting, and that the method of the invention, including its applications, may be varied without departing from the scope of the invention.

What is claimed is:

1. A system for providing suction-assisted surgery with a reduced length of tubing, comprising:
a body tube connected to a suction source;
two extremities tubes;
a Y-shaped connector for connecting the two extremities tubes to the body tube;
a first suction-utilizing surgical instrument or device connected to a first of the two extremities tubes; and
a second suction-utilizing surgical instrument of device connected to a second of the two extremities tubes.

2. A system as claimed in claim 1, wherein the first and second suction-utilizing surgical instruments or devices are arthroscopic surgical instruments or devices.

3. A system as claimed in claim 2, further comprising:
a second body tube connected to a suction source;
two additional extremities tubes;
a second Y-shaped connector for connecting the two extremities tubes to the body tube;
a third suction-utilizing surgical instrument or device connected to a first of the two additional extremities tubes; and
a fourth suction-utilizing surgical instrument or device connected to a second of the two additional extremities tubes

4. A system as claimed in claim 3, wherein the first suction-utilizing surgical instruments is an arthroscopic shaver and the second suction-utilizing surgical instrument is at least one of an arthroscopic cautery and a camera.

5. A system as claimed in claim 4, wherein the third suction-utilizing device is a fluid waste collection bag and the fourth suction-utilizing device is a floor collector.

6. A system as claimed in claim 3, wherein the third suction-utilizing device is a fluid waste collection bag and the fourth suction-utilizing device is a floor collector.

7. A system as claimed in claim 2, wherein the first suction-utilizing device is a fluid waste collection bag and the second suction-utilizing device is a floor collector.

8. A system as claimed in claim 1, wherein the first and second suction-utilizing surgical instruments or devices are instruments or devices used in at least one of the following procedures: a major arthroscopic procedure involving a knee, hip, or shoulder, a minor arthroscopic procedure involving wrist, ankle, or hand, ACL and PCL reconstruction, cystoscopy, spinal surgery, and a laparoscopic procedure

9. A method of providing suction during a surgical procedure, comprising the steps of:
providing a Y-configured suction hose having a body tube connected to a pair of extremities tubes by a Y-connector;
connecting the body tube to a suction port of a suction canister;
connecting a first of the extremities tubes to a first surgical instrument or device;
connecting a second of the extremities tubes to a second surgical instrument or device; and
performing the surgical procedure while providing simultaneous suction to the first and second instruments or devices through the extremities tubes and the body tube.

10. A method as claimed in claim 9, further comprising the steps of:
providing a second Y-configured suction hose having a second body tube connected to a second pair of extremities tubes by a second Y-connector;
connecting the second body tube to a suction port of the suction canister;
connecting a first of the second pair of extremities tubes to a third surgical instrument or device;
connecting a second of the second pair of extremities tubes to a fourth surgical instrument or device; and
performing the surgical procedure while providing simultaneous suction to the first, second, third and fourth instruments or devices through the two pairs of extremities tubes and the two body tubes.

11. A method as claimed in claim 10, wherein the surgical procedure is an arthroscopic surgical procedure.

12. A method as claimed in claim 11, wherein the first and second surgical instruments or devices include at least one of an arthroscopic shaver, an arthroscopic cautery, and a camera, and the third and fourth surgical instruments or devices include at least one of a bag collector and a floor collector for waste fluids.
